(19) United States
(12) Patent Application Publication
(10) Pub. No.: US 2008/0045448 A1
(43) Pub. Date: Feb. 21, 2008

(54) REVERSING AUTONOMIC NERVOUS SYSTEM DYSFUNCTION BY POTENTIATING METHYLATION

(76) Inventor: Alan Robert Vinitsky, Potomac, MD (US)

Correspondence Address: INTELLECTUAL PROPERTY STRATEGISTS, LLC, 7908 CHARLESTON CT., BETHESDA, MD, 20817, US

(21) Appl. No.: 11/465,597

(22) Filed: Aug. 18, 2006

(51) Int. Cl. A61K 31/714; A61K 38/14; A61K 31/525

(52) U.S. Cl. 514/8; 514/52; 514/250; 514/251

(57) ABSTRACT

The present invention is a method and composition for reversing dysfunction of the human autonomic nervous system. The invention consists of administering a methylation-promoting composition that promotes uninterrupted recycling of homocysteine to methionine and uninterrupted processing and removal of metabolic products of stress.

### FIELD OF THE INVENTION

The present invention pertains to methods and compositions for reversing dysfunction of the human autonomic nervous system by administration of methylation-promoting compositions. In particular, the present invention describes a method based on administration of compositions that promote uninterrupted recycling of homocysteine to methionine and uninterrupted processing and removal of metabolic products of stress.

### BACKGROUND OF THE INVENTION

The autonomic nervous system (ANS), including branches of the sympathetic nervous system (SNS) and the parasympathetic nervous system (PSNS), terminates the stress response and regulates bodily functions. Generally, the stress response involves activation of the SNS and requires energy expenditure, whereas termination of the stress response often occurs via the PSNS which is calming, healing and energy-conserving. PSNS is also responsible for regulating most bodily functions [Vinitsky, A. R. Putting the pieces together, Healthy Aging, 1, (5), 2006, pp 63-66].

The ability to methylate via the methionine cycle is available in every cell [Finklestein, J. D. The metabolism of homocysteine: pathways and regulation, European Journal of Pediatrics, 157 (Suppl. 2), 1998, pp. S40-S44]. Methylation processes can be ranked (identified as the Methylation Priority Principle—MPP as given in Table 1.) according to the body's immediate needs for survival—alert for stress, rid the body of stressor(s), reverse the alert response when the stress is cleared, maintenance of bodily functions, repair and healing, and last, energy storage [Vinitsky, A. R. Parts of the puzzle, Healthy Aging, 1(6), 2006, pp. 75-78]. When the resources for methylation are limited, those functions that are not immediately necessary for survival or stress reversal will be reduced or suspended. The consequences of this principle account for the evolution of regional and generalized autonomic dysfunction. Acute and chronic medical conditions and symptoms then develop, resulting from inability to effectively regulate autonomic functions, failure to repair and heal, grow and store energy. In other words, less important methylations can and will be deferred until stress is resolved.

Ideally, the body's response to stress should be immediate, brief (but in no instance longer than necessary) and with the appropriate magnitude. Likewise, termination of the stress response should occur at the right time and with the appropriate magnitude. The end result of these interactions should be a return to the previous state of health. By definition, optimal health is that ideal state of ANS responsiveness to regulate stress responses.

Less than ideal immediate stress responses result in adaptive changes and metabolic consequences. These may include lipid peroxidation [Thiele G., Duryee M. J., Willis M. S., Sorrell M. F., Freeman T. L., Tuma D. J., and Klassen L. W. Malondialdehyde-acetaldehyde (MAA) modified proteins induce pro-inflammatory and pro-fibrotic response by liver endothelial cells, Comparative Hepatology, 3 (Suppl 1), 2004, p. S25)] and the production of nitric oxide via the inducible nitric oxide synthase pathway [Pall, M. Elevated nitric oxide/peroxynitrite theory of multiple chemical sensitivity: central role of N-methyl-D-aspartate receptors in the sensitivity mechanism, Environmental Health Perspectives, 111(12), 2003, pp. 1461-1464]. Sustained stress results in excess production of stress metabolites and depletion of necessary protective molecules [Brouwer M., Chamulitrat W., Ferruzzi G., Sauls D. L., Weinberg J. B. Nitric oxide interactions with cobalamins: biochemical and functional consequences, Blood, 88(5), 1996, pp. 1857-1864]. For example, during the inflammatory phase of their respiratory distress, asthmatics exhale increased levels of aldehydes (Adh) and decreased levels of glutathione (GSH). Following treatment with prednisone, Adh declines and GSH rises [Corradi M., Folesani G., Andreoli R., Manini P., Bodini A., Piacentini G., Carraro S., Zanconato S., Baraldi E. Aldehydes and glutathione in exhaled breath condensate of children with asthma exacerbation, American Journal of Respiratory Critical Care Medicine, 167, 2003, pp. 395-399].

Methylation (Table 1) is required in the body's generalized response to stress and its termination of the response. For example, heightened and sustained stress alerting requires adrenal responses to produce additional adrenalin and excess SNS activity. Adrenalin is activated by methylation. In turn, adrenalin must be inactivated by methylation. Increased PSNS activity to counterbalance SNS activity requires additional production of acetylcholine (Ach), which requires methylation for its production.

TABLE 1
Methylation Priority Principle - MPP.

| FUNCTION | METHYLATION FUNCTION |
|---|---|
| Alert for Stress | Activate ADRENALIN, increase |
| | sympathetic activity |
| Reverse the Alert for Stress | Inactivate ADRENALIN. Increase |
| | parasympathetic activity; make |
| | choline for ACETYLCHOLINE |
| Remove the Stress or the | |
| response to Stress | |
| Allergy - Histamine release | Inactivate HISTAMINE |
| Metals - mercury, arsenic, | Metabolize for excretion |
| tin, selenium & others | |
| Toxins | Metabolize for excretion |
| Niacin | Inactivate |
| Estrogen | Metabolize |
| Maintenance of bodily functions | Inactivate DOPAMINE, |
| (examples): | NOREPINEPHRINE and |
| Blood pressure, pulse | SEROTONIN, |
| Breathing | depending on needs of |
| Bowel habits | autonomic function |
| Urine habits | |
| Mood, memory, concentration, | |
| balance, movement | |
| Sleep | Activate MELATONIN; inactivate |
| | NOREPINEPHRINE more and |
| | SEROTONIN less. |
| Repair and Heal | CREATE RNA for protein |
| | synthesis. Proteins for - |
| | growth - |
| | remodeling (skin, bone, gut |
| | lining) - |
| | enzymes for bodily functions |
| | REPAIR DNA |
| Energy Storage | Form CREATINE |

The Methylation Priority Principle (MPP) indicates that methylation resources of the body are allocated by higher priority toward stress responses at the expense of routine bodily functions, whenever there are inadequate resources to sustain methylation for all functions. Consequently, there will be gradual or abrupt onset of symptoms of body malfunction that may be transient, intermittent, prolonged or progressive.

The following symptoms are features of ANS dysfunction in its earliest stages of evolution: (1) localized or regional dysfunction—pain, temperature or vascular dysregulation; organ dysmotility, such as gastrointestinal and genitourinary; cardiovascular dysfunction—blood pressure alterations and pulse irregularities; neuro—motor, sensory, cognitive or psychosocial dysfunctions; hematologic, immunologic, dermatologic or secretory dysfunctions; (2) generalized dysfunction—multiple, regional dysfunctions occurring simultaneously, in stages, sequences or complex patterns.

Examples of regional dysfunction include so-called vasomotor rhinitis, palpitations, tics, constipation or urinary urgency. Generalized dysfunctions may include such end-stage autonomic dysfunction as syncope, and postural tachycardia. Well-recognized medical conditions [Toth C, and Zochodne D W., Seminars in Neurology 23(4), pp. 373-380, (2003)] often have autonomic neuropathy as a manifestation, such as diabetes, multiple sclerosis and Parkinson syndrome.

Prolonged decompensated stress—intermittent or continuous—is frequently unrecognized. Those individuals with undetected stress may suddenly experience a serious morbidity, a life-threatening or life-ending event. They have undisclosed ANS dysfunction, just as those who present with ongoing symptoms.

The consequence of prolonged stress is failed or inadequate methylation, resulting in ANS dysfunction, and ultimately failure to repair, heal and store energy. Failure to repair and heal results in failed protein synthesis, furthering bodily malfunction, such as enzyme synthesis for complete digestion. Incomplete digestion results in reduced availability of essential amino acids, essential fats and carbohydrates, thereby exacerbating ANS decline.

Status of ANS activities can be directly measured. One computerized diagnostic technique was developed by “The Ansar Group, Inc”, of 240 South 8^{th} Street, Philadelphia, Pa. The ANSAR® technique simultaneously assesses PSNS and SNS functions by digitally monitoring and recording respiratory and heart rates during rest and challenges specially designed to affect SNS and PSNS branches in contrasting ways. By performing real time digital bi-spectral analysis of recorded signals the ANSAR® program can directly measure spectral powers in different frequency domains of heart rate signals and use it for quantitative evaluation of activities of the SNS and PSNS branches of ANS.

### DESCRIPTION OF THE PRIOR ART

Many practitioners of the art of diagnosis and treatment of diseases realized the importance of the methylation process and methylation-promoting substances. Prior art describes many examples of administration of methylation-promoting substances for treatment and monitoring of a variety of isolated medical conditions. However, prior art fails to consider two important characteristics of human function—1) the hierarchy of methylation processes according to the MPP (Table 1.) and 2) the imperative role of the ANS in diagnosing, optimizing treatment, and monitoring patients' conditions. As a consequence, disclosures of the prior art generally omit important interdependences of conditions and symptoms in patients with complex, frequently chronic, systemic diseases and conditions.

Some examples of methylation treatment and diagnostics methods and methylation-promoting substances of the prior art are given below:

U.S. Pat. No. 6,863,906 to Henderson et al. (continuation-in-part of U.S. Pat. No. 6,555,141) discloses methods and therapeutic compositions for treatment and repair of liver tissue, containing S-Adenosylmethionine, L-Ergothioneine, constituents of milk thistle, silymarin and combinations.

U.S. Pat. No. 6,773,892 to Deth, discloses a method of identifying an agent for treating schizophrenia or a related neuropsychiatric disorder by detection of modification of dopamine D4 receptor-linked phospholipid methylation.

U.S. Pat. No. 6,583,123 to Henderson et al. discloses compositions for treatment and repair and for reducing the inflammation of connective tissue in humans and animals comprising S-Adenosylmethionine in combination with fragments of a glycosaminoglycan selected from the group consisting of chondroitin and chondroitin salts.

U.S. Pat. No. 6,555,141 to Henderson et al (CiP of U.S. Pat. No. 6,555,141) describes therapeutic compositions containing S-Adenosylmethionine, L-Ergothioneine, constituents of milk thistle, silymarin and combinations for the protection, treatment and repair of liver tissue.

U.S. Pat. No. 6,129,918 to Amagase describes a method and composition for reducing the elevated plasma homocysteine level in mammals by oral administration of a therapeutically effective amount of Allium plants or extracts thereof, preferably garlic, with or without one or more vitamins such as B₆, B₁₂ and folic acid in an acceptable pharmaceutical carrier.

U.S. Pat. No. 6,121,249 to Weissman et al. discloses a method for treatment of cardiovascular diseases and “a daily administration pack” containing antioxidants, cyanocobalamin, folic acid, pyridoxine and niacin compounds.

U.S. Pat. No. 5,795,873 to Allen discloses a method of treating or preventing elevated serum metabolite levels of at least one of homocysteine, methylmalonic acid, cystathionine, or 2-methylcitric acid comprising a periodically orally administered single formulation having between 0.3-10 mg vitamin B₁₂ and 0.1-0.4 mg folic acid.

US Patent Application Pub. #20060063786 to Mueller et al. describes an invention which relates to the use of folates for producing a pharmaceutical preparation suitable for the prevention and treatment of inflammation and diseases associated with inflammation, particularly for influencing the inflammation markers C-reactive protein and serum amyloid A protein.

A set of related US Patent Applications (Pub. #20060034954, 20040220118, 20030190381, and 20020192310) to Bland et al. discloses a method of managing hormone imbalances like estrogen imbalance and alleviations of related symptoms achieved by administration of dietary supplements comprised of a mixture of an isoflavone, an isoflavone synergist and a methylation support compound.

A separate portion of the background prior art related to the current invention comprises publications that disclose general methods and compositions for treatment of deficiencies in B-complex vitamins, especially B₁₂ (cobalamins), B₆ (pyridoxine) and folates.

U.S. Pat. No. 6,369,041 to Horrobin et al. discloses an orally administrable formulation containing hydroxocobalamin and folic acid or a related bioactive derivative of folic acid for treatment of vitamin B₁₂ deficiencies.

US Patent Application Pub. #20050143340 to Collins discloses “a fortified food composition comprising a fortifying amount of adenosylcobalamin, mixed with or bound to intrinsic factor, transcobalamin I, transcobalamin II and/or transcobalamin III, wherein the food composition is selected from the group consisting of a cereal, a gelled confection consisting primarily of sugars and a fruit base, a chewing confection, a cereal bar or granola bar, a supplement, a fruit juice, a vegetable juice, a botanical juice, popcorn, pretzels, nuts, potato chips, and fries.”

US Patent Application Pub. #20040157783 to McCaddon discloses “a medical composition for use in the treatment and/or prevention of a functional Vitamin B₁₂ deficiency that occurs due to a disorder in the intracellular processing of Vitamin B₁₂ rather than the malabsorption thereof, the symptoms of said functional Vitamin B₁₂ deficiency including elevated blood levels of homocysteine and/or methylmalonic acid, and/or low levels of total serum B₁₂ and/or low levels of holo-transcobalamin, the composition comprising a compound or combination of compounds that directly or indirectly supply a cobalt-sulphur bond in the upper β-axial ligand of an intracellular cobalamin molecule thereby facilitating intracellular processing of cobalamins.”

In addition to prior art in patents and patent applications, PERQUE LLC, of Sterling, Va. (14 Pidgeon Hill Drive, Suite 180, Sterling, Va. 20165) produces and distributes through medical professionals a family of dietary supplement products containing hydroxocobalamin-folic acid combinations. In particular, PERQUE Vessel Health Guard™ product is advertised as a treatment against blood vessel hardening contains 10 mg of B6, 2 mg of B12, 2.5 mg of folic acid, 53 mg of Magnesium, 198 mg Mannitol, 10 mg of whole cherry fruit extract, 2 mg of Sucanat® and 10 mg of Xylitol per single sublingual lozenge. (www.perque.com/product-info.asp as reviewed Jul. 25, 2006)

### SUMMARY OF THE INVENTION

The present invention provides methods and compositions for reversing dysfunction of the human autonomic nervous system by administration of methylation-promoting compositions. Folic acid—its folate salts—and cobalamin are the critical nutrients at the intersection of methylation and ANS balance. They must be administered simultaneously for an immediate effect to occur. Symptom relief is often noted in minutes, depending on number of individual doses required. The route of administration is also critical, as delays in absorption and the interaction of cobalamin with its carrier protein will interrupt any immediate relief of acute symptoms.

The present invention therefore addresses the need to replenish folic acid and cobalamin in an optimal form and optimal ratio, which will result in reversal or prevention of inadequate methylation and resulting ANS dysfunction.

### BRIEF DESCRIPTION OF THE DRAWINGS

FIG. 1 is a simplified schematic diagram of basic biochemical processes on which the current invention is based.

FIG. 2 is a graphic representation of (a) unbalanced ANS before the treatment and (b) reversal of the condition achieved by the treatment in accordance with the preferred embodiment of the present invention.

Like reference numerals identify like parts throughout the drawings.

### DETAILED DESCRIPTION OF THE INVENTION

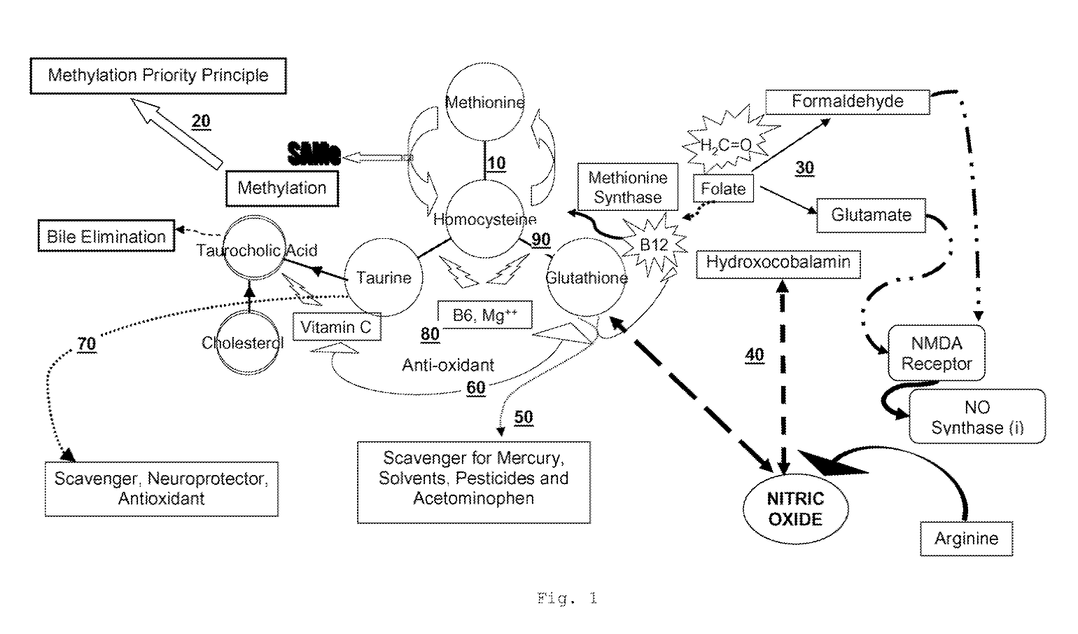

FIG. 1. schematically represents basic biochemical processes on which the current invention is based. The schematic diagram in FIG. 1. is simplified to demonstrate significant relationships between compounds used in compositions and methods for reversing dysfunction of the human autonomic nervous system in the current invention. Consequently, the schematic in FIG. 1. is not a representation of the mechanism of action of disclosed compounds and methods.

The methylation cycle 10, Methionine→S-Adenosylmethionine→S-Adenosylhomocysteine (denoted by standard abbreviation SAMe)→Homocysteine→(remethylation involving hydroxocobalamin and folate) back to Methionine in FIG. 1. is a part of normal cell function well known in prior art. For example, U.S. Pat. No. 6,583,123 to Henderson et al. discloses use of S-Adenosylmethionine (SAMe) for treatment and for reducing inflammation of connective tissue.

During a reaction to stress situations, demand for methyl group donors (like SAMe) suddenly increases, in accordance with the Methylation Priority Principle 20, driven by the need to perform additional “Methylation Functions” (Table 1.) Specific stressors requiring methylation further exacerbate the stressed state, thereby destabilizing the autonomic nervous system.

Metabolically active folates like 5-methyltetrahydrofolate can be created in the body via a complex process known as “liver metabolism” from dietary folic acid (vitamin B9)—complex mixtures of polyglutamate (multiple glutamate molecules attached) conjugate compounds. For the purpose of this invention, dietary supplements denoted as first compound, in less complex forms of folic acid, folates, folinic acid and its salts—folinates (like calcium folinate known as leucovorin calcium) that do not need liver metabolism for transformation to methyltetrahydrofolate are preferred. However, the dihydrofolate form functions exceptionally efficiently as a scavenger for aldehydes and glutamate, hence should be considered, even if it needs to be administered as an active component.

Similarly, supplement denoted as second compound—aquacobalamin, methylcobalamin, glutathionylcobalamin and adenosylcobalamin—biochemically close to the metabolically active hydroxocobalamin (OH—B12) are preferred for the current invention.

Folate scavenges 30 for aldehydes and glutamate and recycles aldehydes through hydroxocobalamin to homocysteine. Hydroxocobalamin is reduced by glutathione but also scavenges for nitric oxide (NO) 40 and peroxynitrite. Hydroxocobalamin can be inactivated by NO. Aldehydes, NO and peroxynitrite, if allowed to accumulate, can severely disturb balance and functions of both branches of the ANS.

Glutathione (GSH) scavenges 50 for mercury, pesticides and solvents and metabolizes acetominophen. Glutathione can be inactivated by NO, can reduce ascorbate (vitamin C) or can be reduced 60 by it. Taurine scavenges 70 for hypochlorite and other chlorine-containing compounds. Taurine, magnesium and pyridoxine (vitamin B6) encourage the production of glutathione when homocysteine is converted to cysteine 90.

Oxidative stress leads to increased aldehydes, nitric oxide and oxidized glutathione (or less available reduced GSH). Nitric oxide causes increased pain and inflammation through the inducible nitric oxide synthase pathway, which is potentiated by formaldehyde, glutamate and solvents.

When nutrients are diverted for scavenging or are inactivated alone or in combination, methylation will become inadequate under stress. If hydroxocobalamin is insufficient or dysfunctional, then methylation cannot proceed. When GSH is insufficient or dysfunctional, hydroxocobalamin is dysfunctional. In either instance, folate becomes functionally insufficient, even when levels are elevated, because it is saturated with methyl groups and aldehydes. This condition is called the folate trap.

The schematic in FIG. 1. infers that key methylation-promoting substances, reduced hydroxocobalamin and methyltetrahydrofolate must be simultaneously available in proper ratio to alleviate stress-induced imbalances of ANS caused by increased demands for methylation. In order to efficiently relieve symptoms of ANS imbalance, it is desirable to administer methylation-promoting compounds as proper mixes of respective metabolically active forms, i.e. hydroxocobalamin and methyltetrahydrofolate, or in forms closely related to respective metabolically active forms like folates, folinic acid, folinates; aquacobalamin, methylcobalamin, glutathionylcobalamin, adenosylcobalamin and their mixtures and combinations.

Method of administration should be chosen to circumvent the gastrointestinal tract and first-pass liver metabolism and allows methylation-promoting compounds to directly enter the human body circulation allowing for fast and simultaneous occurrence of high tissue concentrations of all methylation-promoting components. This requirement makes oral and intramuscular administration methods the least favorable because of long and uncontrollable delays between the time of administration and the time to achieving maximal tissue concentrations. Direct intravenous or subcutaneous administrations, although obviously effective, are inconvenient due to the impracticality of long-term self-administration. However, intermittent intravenous therapy can be a useful therapeutic intervention.

The route of administration of methylation-promoting compositions through mucous membranes (transmucosal) to the proximal circulation pathways satisfies requirements for effective simultaneous delivery. Mucous membranes of the buccal cavity offer convenient and efficient access of transbuccally administered compositions to the body circulation. It is conceivable that inhaled (via nebulizer), intranasal or subcutaneous routes may be options, but folic acid has not been sufficiently optimized and tested for administration via these routes. Inconvenient as they are, transrectal and transvaginal routes should also be effective.

Transdermal application, despite lower permeability and maximum achievable tissue concentration relative to transmucosal administration, may possible be a desirable and effective route of administration for certain ANS balance maintenance and “stress prevention” treatments.

Folates (folinic acid, folinic acid and folinates) and cobalamins (hydroxocobalamin, aquacobalamin, methylcobalamin, glutathionylcobalamin and adenosylcobalamin) are known dietary supplements available commercially in a liquid (solutions), powder or tablet form. Because of a wide variation in optimized single doses and possibility for progressive improvements in doses and ratios of active ingredients, it is frequently desirable to store folates and cobalamins separately and combine them into a particular methylation-promoting composition just prior to the time of simultaneous administration. It is possible that stable fixed-dose combinations of the invention can be developed.

It is evident from Table 1. and FIG. 1. that methylation-promoting compositions and compounds participate in a multitude of frequently competing methylation processes. Because of composition's complicated metabolism, single unit doses and total daily doses can vary significantly, most frequently as a function of a) individual symptoms, b) relief of symptoms, c) wear-off effect, (i.e. return of symptoms), d) adverse reactions or side effects of a treatment dose and e) prophylactic dosing. Listed factors further depend on patients' individual conditions, such as severity and frequency of exposures to stressors, age, general state of fitness, diet, rest, etc.

Single unit dose of said first compound ranges from 0.5 mg to 50 mg. Single unit dose of said second compound ranges from 0.2 mg to 20 mg. Single unit dose mass ratio of said first compound versus said second compound ranges from 0.5 to 5.

Depending on individual dosing and treatment schedule, the cumulative daily dose is likely to change, possibly daily. During applications of methods in accordance with the present invention total daily dose did not exceed 200 mg of said first compound and 80 mg of said second compound. Similarly, total number of administered unit doses per day did not exceed 40 unit doses for adult and 30 unit doses for children 13 years of age or younger.

Patients' monitoring did not indicate contraindications associated with doses consisting of upper limits of listed ranges. Even higher doses are considered applicable to some particularly severe conditions of ANS imbalance at least for short duration as part of a prolonged regimen.

Inspection of schematics in FIG. 1. indicates that methylation-promoting functions require vitamin C 60 and B6-Mg++ combination 80. If those ions and compounds are deficient, the methylation-promoting composition comprising said first compound and said second compound would not be effective, regardless of the dosing and administration method. It is in accordance with the current invention to supplement the deficient additives together with said first and second compound. The methylation-promoting composition in accordance with this invention was successfully combined with multiple vitamin and mineral supplements containing B complex vitamins, fat-soluble vitamins, magnesium, zinc, biotin and their mixtures and combinations.

Performance of “methylation functions” in Table 1. is contingent on the availability of multiple enzymes, cofactors and dietary supplements. If those compounds are not sufficiently available, one needs to supplement them as additions to the methylation-promoting composition comprising said first compound and said second compound. The methylation-promoting composition comprising said first compound and said second compound can be successfully supplemented by taurine, glutathione reduced, essential amino acids, essential fatty acids, anti-oxidants, calcium, iron, copper, selenium, chromium, vanadium, manganese, molybdenum, boron, iodine/iodide, phosphorus/phosphate, phospholipids, dimethylaminoethanol, inositol, dimethylglycine, betaine, gamma-amino butyric acid, natural hormone replacements, digestive enzymes and probiotics and their mixtures and combinations. Other compounds and dietary supplements can also be added without exceeding the scope of the present invention.

### DESCRIPTION OF PREFERRED EMBODIMENT

From the detailed description of the invention it follows that optimal application of the methylation-promoting composition strongly depends on the circumstances and conditions characteristic of each individual patient. Nevertheless, for the majority of patients optimum dosing contains first compound and second compound with a mass ratio approaching 5/2 with no preservatives and free of excipients that potentially affect diuresis (such as mannitol) or significantly stimulate the ANS adversely.

Proffered method of administration is transbuccal wherein methylation-promoting composition consists of a solution comprising 5 mg of folic acid applied to a 2 mg tablet of hydroxocobalamin administered simultaneously on arising, midday and at bedtime. Folirinse™ (10% Folate Solution, 5 mg/drop) by Scientific Botanicals, Inc. and Activated B-12 Guard™ (2000 mcg Sublingual Lozenges) by Perque LLC are used successfully as the preferred choices.

Usual wear-off time is approximately three hours after a dose. If this phenomenon is observed, two or more doses can be administered concurrently to restore a three times a day schedule.

Situations (with autonomic symptoms or prophylaxis of same) may arise that will dictate more frequent dosing than the three times a day schedule.

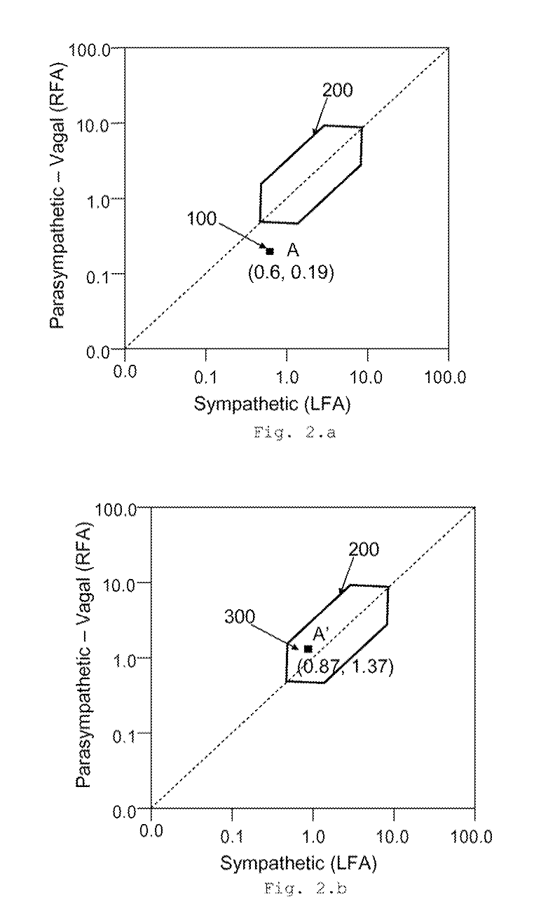

Effectiveness of the therapy based on the preferred embodiment is illustrated in FIG. 2.a and FIG. 2.b. Using direct examination, questionnaire, and ANSAR® technique of simultaneously assessing PSNS and SNS functions, severe imbalance of ANS (autonomous neuropathy) is diagnosed. The severity of the condition can be observed from FIG. 2.a obtained before the treatment. FIG. 2.a describes overall balance of ANS by graphing integral spectral power in low frequency area (LFA) of patient's “at rest” heart rate signal (measure of sympathetic activity) versus spectral power in respiratory frequency range (RFA) (measure of parasympathetic—vagal activity). Measured point A 100 at 0.60, 0.19) coordinates indicates significant imbalance exhibiting LFA/RFA ratio of 3.10. Measured point A 100 is significantly outside the acceptable region 200 even when plotted on relatively insensitive logarithmic scales.

FIG. 2.b represents an analogous graph recorded after treatment in accordance with the preferred embodiment. It is immediately noticeable that measured response A′ 300 moved to coordinates (0.87, 1.37) exhibiting LFA/RFA ratio of 0.63. The after-treatment coordinates approach the ideally balanced (1, 1) point and LFA/RFA ratio of 0.63 nears the perfect value of 1. These measurements are considered very satisfactory. The most dramatic improvement is the increase of RFA value from 0.19 to 1.37 which represents more then 720% improvement of PSNS activity. Even more significant is the ability to maintain the desirable improvements for years. In other words, the preferred treatment improved the power of the healing, energy-conserving PSNS and reversed the excessive dominance of the energy-expending SNS.

We claim:

1. A method for reversing dysfunction of the human autonomic nervous system by administration of a methylation-promoting composition that promotes uninterrupted recycling of homocysteine to methionine and uninterrupted processing and removal of metabolic products of stress.

2. The method for reversing dysfunction of the human autonomic nervous system of claim 1. wherein the methylation-promoting composition comprises
first compound chosen from a group of compounds consisting of folic acid, folates, folinic acid, folinates, dihydrofolate, methyltetrahydrofolate and their mixture and combinations; and
second compound chosen from a group of compounds consisting of hydroxocobalamin, aquacobalamin, methylcobalamin, glutathionylcobalamin, adenosylcobalamin and their mixtures and combinations.

3. The method for reversing dysfunction of the human autonomic nervous system of claim 1. wherein methylation-promoting composition is free of preservatives, excipients that affect diuresis and excipients that directly effect autonomic nervous system.

4. The method for reversing dysfunction of the human autonomic nervous system of claim 1. wherein administration of a methylation-promoting composition is performed using an administration method that avoids the gastrointestinal tract and first-pass of liver metabolism and allows methylation-promoting composition to directly enter into the human body circulation.

5. The administration method of claim 4. is a transdermal administration method.

6. The administration method of claim 4. is an intravenous administration method.

7. The administration method of claim 4. is a subcutaneous administration method.

8. The administration method of claim 4. is a transmucosal administration method, said transmucosal administration method includes transbuccal, intranasal, inhaled, transrectal and transvaginal administration methods.

9. The method for reversing dysfunction of the human autonomic nervous system of claim 2. wherein said first compound and said second compound are stored separately and combined into said methylation-promoting composition just prior to the time of simultaneous administration.

10. The method for reversing dysfunction of the human autonomic nervous system of claim 9. wherein said first compound and said second compound are stored separately in state of liquid, state of powder or tablet form.

11. The method for reversing dysfunction of the human autonomic nervous system of claim 2. wherein single unit dose for said first compound ranges from 0.5 mg to 50 mg.

12. The method for reversing dysfunction of the human autonomic nervous system of claim 2. wherein single unit dose for said second compound ranges from 0.2 mg to 20 mg.

13. The method for reversing dysfunction of the human autonomic nervous system of claim 2. wherein single unit dose mass ratio of said first compound versus said second compound ranges from 0.5 to 5.

14. The method for reversing dysfunction of the human autonomic nervous system of claim 2. wherein total daily dose does not exceed 200 mg of said first compound and 80 mg of said second compound.

15. The method for reversing dysfunction of the human autonomic nervous system of claim 2. wherein the total number of administered unit doses per day does not exceed 40 unit doses for adult and 30 unit doses for children 13 years of age or younger.

16. The method for reversing dysfunction of the human autonomic nervous system of claim 2. wherein, in addition to said first compound and said second compound, said methylation-promoting composition comprises compounds chosen from a group consisting of multiple vitamin and mineral supplements containing B complex vitamins, fat-soluble vitamins, magnesium, zinc, biotin and their mixtures and combinations.

17. The method for reversing dysfunction of the human autonomic nervous system of claim 2. wherein, in addition to said first compound and said second compound, said methylation-promoting composition comprises compounds chosen from a group consisting of taurine, glutathione reduced, essential amino acids, essential fatty acids, anti-oxidants, calcium, iron, copper, selenium, chromium, vanadium, manganese, molybdenum, boron, iodine/iodide, phosphorus/phosphate, phospholipids, dimethylaminoethanol, inositol, dimethylglycine, betaine, gamma-amino butyric acid, natural hormone replacements, digestive enzymes and probiotics, and their mixtures and combinations.

18. The method for reversing dysfunction of the human autonomic nervous system of claim 2. wherein methylation-promoting composition consists of a solution comprising 5 mg of folic acid applied to a 2 mg tablet of hydroxocobalamin administered by a transbuccal administration method on arising, midday and at bedtime.

19. The methylation-promoting composition for treatment of dysfunction of the human autonomic nervous system that promotes uninterrupted recycling of homocysteine to methionine and uninterrupted processing and removal of metabolic products of stress, comprises
first compound chosen from a group of compounds consisting of folic acid, folates, folinic acid, folinates, dihydrofolate, methyltetrahydrofolate, and their mixtures and combinations; and
second compound chosen from a group of compounds consisting of hydroxocobalamin, aquacobalamin, methylcobalamin, glutathionylcobalamin, adenosylcobalamin and their mixtures and combinations.

20. The methylation-promoting composition of claim 19. wherein, in addition to said first compound and said second compound, said methylation-promoting composition comprises compounds chosen from a group consisting of multiple vitamin and mineral supplements containing B complex vitamins, fat-soluble vitamins, magnesium, zinc, biotin and their mixtures and combinations.

21. The methylation-promoting composition of claim 19. wherein, in addition to said first compound and said second compound, said methylation-promoting composition comprises compounds chosen from a group consisting of taurine, glutathione reduced, essential amino acids, essential fatty acids, anti-oxidants, calcium, iron, copper, selenium, chromium, vanadium, manganese, molybdenum, boron, iodine/iodide, phosphorus/phosphate, phospholipids, dimethylaminoethanol, inositol, dimethylglycine, betaine, gamma-amino butyric acid, natural hormone replacements, digestive enzymes and probiotics and their mixtures and combinations.
